(19) United States
(12) Patent Application Publication
(10) Pub. No.: US 2008/0033513 A1
(43) Pub. Date: Feb. 7, 2008

(54) Directed Energy for Point Oriented Medical Treatment

(75) Inventors: Avner MAN, Kibbutz Beit Alfa (IL); Avner AMIR, Ramat Yisahy (IL)

Correspondence Address: SCHWEITZER CORNMAN GROSS & BONDELL LLP, 292 MADISON AVENUE - 19th FLOOR, NEW YORK, NY, 10017, US

(73) Assignee: MedDynamics Ltd., Migdal Haemek (IL)

(21) Appl. No.: 11/632,432

(22) PCT Filed: Jul. 15, 2005
(86) PCT No.: PCT/IL05/00757

§ 371 Date: Jan. 12, 2007

(30) Foreign Application Priority Data
Jul. 15, 2004 (IL) 163060

(51) Int. Cl. A61N 5/06; A61F 7/00; A61F 7/12

(52) U.S. Cl. 607/88; 607/96

(57) ABSTRACT

The present invention discloses a system (1) for administering medical therapy by means of directing energy radiation onto a patient's body. The novel system comprising the following: an assembly for directing said energy in a predetermined measure comprising a means for emitting energy to be directed toward the patient's body, and communications means, a treatment protocol defining a set of parameters according to which the emitted energy is to be delivered, such parameters including predetermined points on the patient's body whereat the energy is to be directed and a system for controlling the emitted radiation according to the protocol. The present invention also discloses a method for administration of laser acupuncture by means of a medical device comprising an emitter of photonic radiation directed to an acupuncture point and an imaging sensor for monitoring and controlling the treatment.

### FIELD OF THE INVENTION

The present invention generally relates to a directed energy treatment system and especially to such a system designed as a medical device for applying methodological medical treatments on specific points on the body such as acupuncture points or trigger points, to a patient.

### BACKGROUND OF THE INVENTION

Acupuncture point therapy, Trigger point therapy (such as described in the book Acupuncture. Trigger Points and Musculoskeletal Pain by P. E. Baldry, 2n Ed. Churchill Livingstone, Edinburgh 1993) and other forms of treatment directed to specific points on the body that are known for medical treatments, hereinafter referred to as acupuncture treatment, and such points as acupuncture points or acupoints. Such methods, though known for many years, provide little if any possibility of quantifying the efficacy of treatment by tools acceptable in modern medical research. These methods have not been researched in depth because of the lack of quantifying, controlling, monitoring, recording data and communicating mechanisms. Such mechanisms, which are essential for developing commonly agreed protocols for modern practice of such methods, are generally lacking. Furthermore, such point-treatments, which though potentially may be dispensed by an expert (man or machine) not in the immediate locality of the patient (hereafter referred as “remote”) have not been so applied for lack of similar research tools.

The book Acupuncture, Trigger Points and Musculoskeletal Pain by P. E. Baldry, 2n Ed. Churchill Livingstone, Edinburgh 1993 describes the methodology of acupuncture as well as the use of trigger points, which are points more familiar to western medical professionals to treat a variety of medical conditions such as pain. This book describes the use of bare needles (p. 97) for stimulating such points for the alleviation of such conditions, the use of application of electricity conducted through the needles to the body (electroacupuncture, pp. 105-106) at such points and the application of alternating current through skin electrodes applied at such points (Transcutaenous Electrical Nerve Stimulation or TENS) (pp. 106-109) for the same purpose. U.S. Pat. No. 6,641,599 to Peterson et al., discloses a method of therapeutic treatment of the patient's body, comprising the steps of: applying a therapeutic modality to a therapeutic micro-system of each of the ear, hand, and foot; stimulating a plurality of therapeutic points of said therapeutic micro-systems; and then therapeutically treating said body in response to said stimulating wherein said stimulating comprises stimulating a plurality of therapeutic points selected from a group consisting of acupressure points, acupuncture points, or meridian points. Such a method has no means of quantifying, controlling, monitoring, recording or analyzing data regarding the treatment and the energy delivered as a therapy during the treatment, and does not include communicating mechanisms for exchange of data with other similar systems. Many devices for acupuncture have been presented in the literature of every day practice. Using laser technology for acupuncture is also known in the art, such as the one presented by U.S. Pat. No. 6,371,954 to Lee. This portable laser device for emitting a laser light comprises a distal housing, and a distal tip, a self-contained power source within said housing, a condensing lens contained within the housing to direct the laser light into and through a hollow ceramic acupuncture maneuverable needle. EP Pat application No. WO0240098 to Schikora teaches a device for performing acupuncture on a patient using laser radiation. Such laser systems have no means of quantifying, monitoring, recording or analyzing data regarding the treatment and the energy delivered as a therapy during the treatment, and does not include communicating mechanisms for exchange of data with other similar systems. Similarly, Japanese Pat. No. 2002/078772 to Kawaguchi provides a video camera installed on a terminal placed in front of the patient at the distant place so that the muscle tonus diagnosis is executed by a sensor added to a personal computer, by collating Western medical materials and related image materials culled from the personal computer while viewing the audio and biological images of the patient transmitted over the Internet. At the same time, the physical therapy based on remote controlled laser acupuncture induced by a CCR zoom camera connected to the terminal in front of the patient can be executed remotely. In the context of the present invention such a unit including a laser (or any energy emitting device for treatment) and a camera (or any monitoring device) is called an “end unit”. The system in Kawaguchi's patent provides a list of end units used to communicate between an expert and a distant patient, and its sole target is to perform a distant treatment by an expert in real time. Such a system has no means of quantifying the amount of laser absorbed by the patient's body, it does not recording or analyze the treatment data or information about the intensity, duration, repetition, wavelength and location of the energy delivered as a therapy during the treatment. This system cannot function as a home-use self administrated medical device.

EAV, i.e., “Electric Acupuncture according to Voll”, after Dr. Reinhold Voll, the originator of that method is a well-known method for building and using an acupuncture end unit while using an Impedance examination of acupuncture points. This method is described in the book “Treatise of Acupuncture” by Charles H. McWilliams, Health Sciences Research, U.S.A. The treatment is prescribed usually using electric stimulation, and/or in conjunction with use of homeopathic or herbal medicines. A wide range of systems using the EAV method exists in the market, but there is no such system able to collect the treatment data and analyze it. Moreover, there is no system able to produce treatment and diagnosis protocols, to be tested and analyzed with the treatment data records, and upgraded by the system. The method of impedance examination is also known to be technically hard to quantify, because of the dependency on achieving good electric contact with the patient skin.

In the context of Acupuncture end units, with possible improvement on the impedance method, the work by Lazoura et al. (2^{nd} International Conference on Bioelectromagnetism, February 1998, Melbourne Australia, pp. 171-172) describes a method of comparing light reflection at acupuncture and non-acupuncture points. The mechanism of detection is bulky and not suitable for general work on any acupuncture point by a therapist or a home-user and further is not suitable as an end unit required under the present invention.

In a report made by MedDynamics Ltd. (Jan. 6, 2004) an experimental setup was built to demonstrate the feasibility of identifying acupuncture points by means of measuring the absorption at the neighborhood of such points. The outcome data of these measurements was calculated and presented in two and three dimensional graphs. A list of acupuncture points was examined (PC4, PC5, PC6, PC7, TW4, TW5, TW6, HT8) compared to their surrounding area.

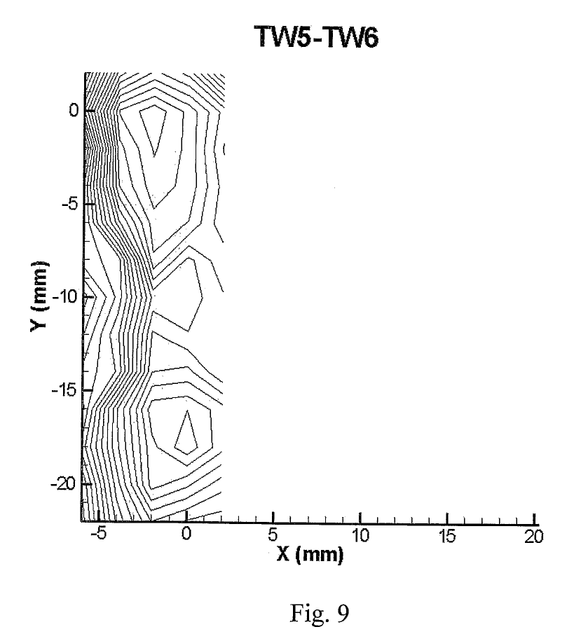

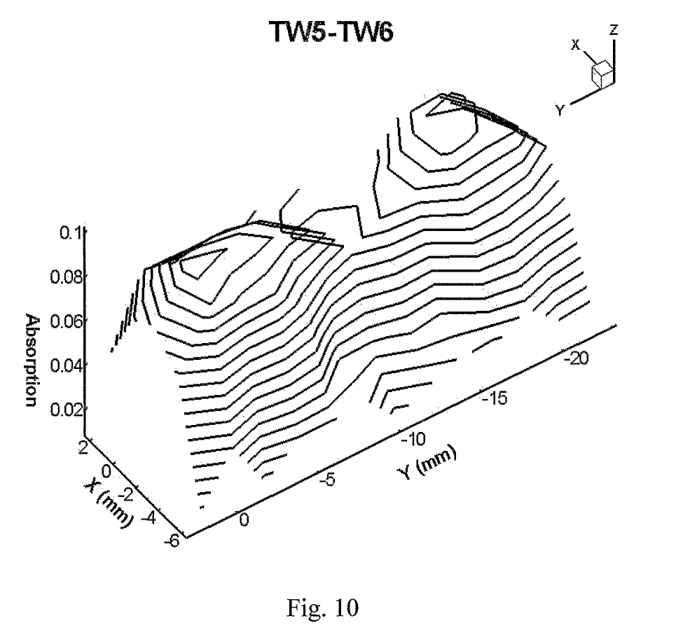

The results presented in the report show a detailed map of absorption near acupuncture points. The results are in general agreement with earlier results obtained by Lazoura et al. The difference here is that Lazoura et al. only investigated a very small number of sampling points and did not conduct extensive mapping as performed under MedDynamics' work, using the more accurate method of integrating spheres. An example of the summary graphs is presented in FIG. 9 (TW5 & TW6 in two dimensions) and FIG. 10 (TW5 & TW6 in three dimensions).

A system that interfaces to an end unit (or multiplicity thereof) of the type disclosed here for administration of medical care is described in a patent application by A Amir and A Man “A System and Method for Administration of Intercommunicated Healthcare” submitted simultaneously with the present application.

### SUMMARY OF THE INVENTION

It is thus the purpose of the present invention to provide a guided medical system for administration of directed energy acupuncture treatment. This system is inter alia comprised of an assembly for directing energy for performing treatment on a patient, said assembly comprising a means for emitting energy to be directed toward the patient's body, communication means, a system of controlling the emitted energy according to a protocol and a protocol defining a set of parameters according to which the energy is to be delivered to the patient's body. Such parameters also include a list of certain points on the patients body where the energy is to be directed.

The energy emitter could be a laser, a light emitting diode, an ultrasound wave generating and directing device or any similar device. In contrast to traditionally used needles or to pressure on acu-points applied manually, the use of such devices allows the application of quantifying, controlling and monitoring mechanisms. The quantifying, controlling and monitoring mechanisms can be used to ensure that the treatment conforms to a certain predefined protocol. The protocol is a set of parameters used in the application of treatment such as intensity, duration, repetition, wavelength and location of treatment. A monitoring device such as a light sensor, a CCD camera or an ultrasound imaging device permits recording of the effect of the energy on the patient and the production of a digital record of the treatment and its effect.

The use of protocols and associated records also allows the communication of results to remote experts. Hereafter we shall refer to Common Medical Information Protocol (CMIP). Such a protocol is essentially a set of data items, such as treatment, diagnosis, communication and other items.

The energy emitter is a part of a means (hereafter called an end-unit) that is used to perform diagnosis and treatment and preferably comprises both means for emitting focused-energy and communication means for transmitting data items necessary for the CMIP. The energy emitter is adapted to emit focused-energy to points in the body as guided by said CMIP so that diagnosis and targeted treatment is provided, monitored, and/or recorded.

It is in the scope of the present invention wherein the CMIP's module is selected from the group of: patient data archiving module; practitioner data archiving module; anamnesis data module; diagnosis protocols data module, diagnosis registration data module; treatment protocols data module; clinical investigation management module; medical knowledge data module; information security data module; registry data module; controlling, monitoring and recording module, data retrieving module or any combination thereof.

Hence, the end unit may additionally comprise at least one sensor adapted for monitoring the body tissue affected by the energy emitted from the device; a unit for analyzing the monitored image and possibly for locating the treatment point and/or measuring the clinical state of a treatment point and/or other purposes; software adapted for collecting the analyzed data from treatment points and further analyzing the data so as to define a medical diagnosis according to a diagnosis protocol from the CMIP; software adapted for using a CMIP defined diagnosis for performing a treatment according to a treatment protocol from the CMIP; a unit for collecting the therapy data for future reviewing, and software for performing statistical analysis on the recorded data so as to improve the diagnosis and treatment protocols in the CMIP.

It is in the scope of the present invention wherein the light, ultrasound, heat or other sort of energy used for treatment and/or diagnosis is transferred directly to the body.

It is also in the scope of the present invention wherein the energy is light transferred to the body by means of an assembly comprising a bundle of fibers adapted to emit light from a light source while conducting the light scattered by the tissue to another part of the bundle and to measure it by a light sensor located on its end furthest away from the tissue.

A second object of the present invention is to present a useful guided method for self-administrated laser acupuncture by means of a medical device comprising an emitter of photonic radiation on an acupuncture point and an imaging sensor for monitoring and controlling the treatment.

It is still in the scope of the present invention wherein the aforesaid method comprises the steps of locating the acupuncture point; measuring the absorption of energy (such as light, ultrasound, heat) at an acupuncture point; determining its functionality, and applying energy-directed treatment to said point; of communicating with a database so that data archiving and/or data retrieving is made possible; of collecting the data from examined treatment points and analyzing the data so as to produce a medical diagnosis; of collecting the therapy data for future reviewing, and for statistic analysis; of collecting and analyzing the data from examined treatment points and the therapy data, according to Clinical Investigation Standards so as to augment the reliability of treatment and diagnosis protocols. The term Clinical Investigation Standard hereby refers to any of the commonly and widely accepted practices used by professionals in performing medical investigation.

It is lastly in the scope of the present invention wherein the said method additionally comprises the step of analyzing light scattered from the tissue for purpose of determining the parameters of the incident radiation and its effect on the body such as the amount absorbed by the body. Such measurement may be used to optimally locate the optimal location of treatment points and/or measuring the clinical state of a treatment point.

### BRIEF DESCRIPTION OF THE INVENTION

In order to understand the invention and to see how it may be implemented in practice, a preferred embodiment will now be described, by way of non-limiting example only, with reference to the accompanying drawing, in which

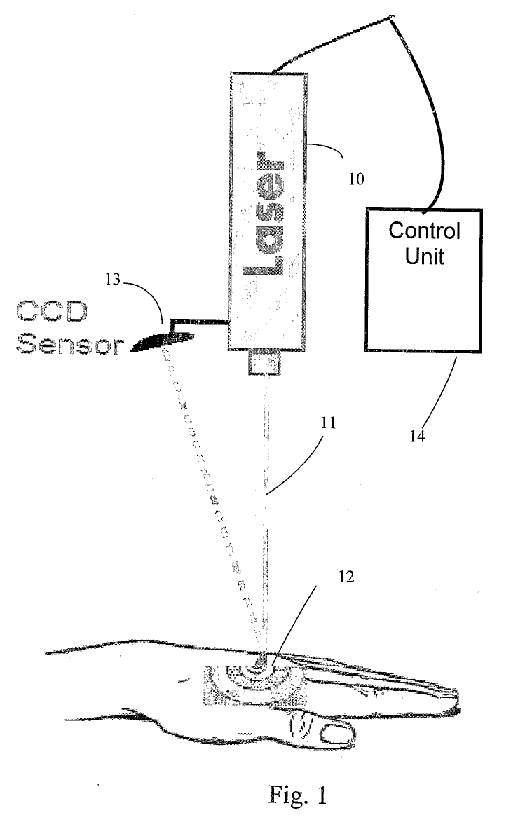

FIG. 1 schematically presents a photonic energy directing treatment system (a laser, for example) creating a detectable irradiated spot on the tissue;

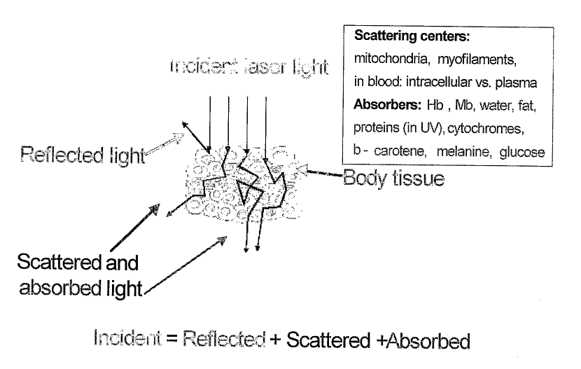

FIG. 2 schematically presents an illustration depicting the energy components hitting the tissue. The interaction of light with the body tissues separates the beam into components to be reflected, scattered or absorbed. The relative amounts of absorption and scattering are measured directly by the method described. The main biological molecules responsible for scattering and absorption are listed in the inset;

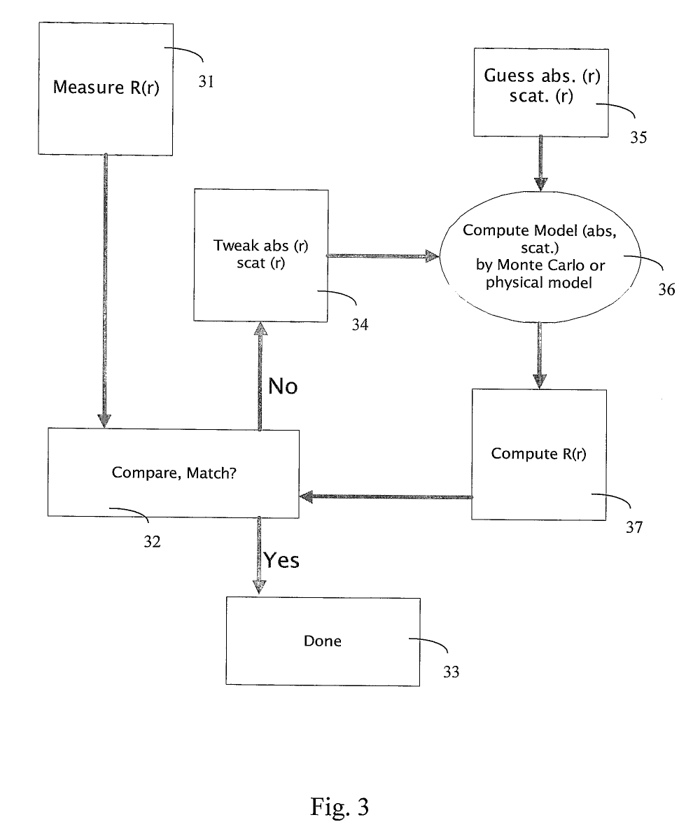

FIG. 3 schematically presents one suitable computational algorithm for computation of the absorption and scattering;

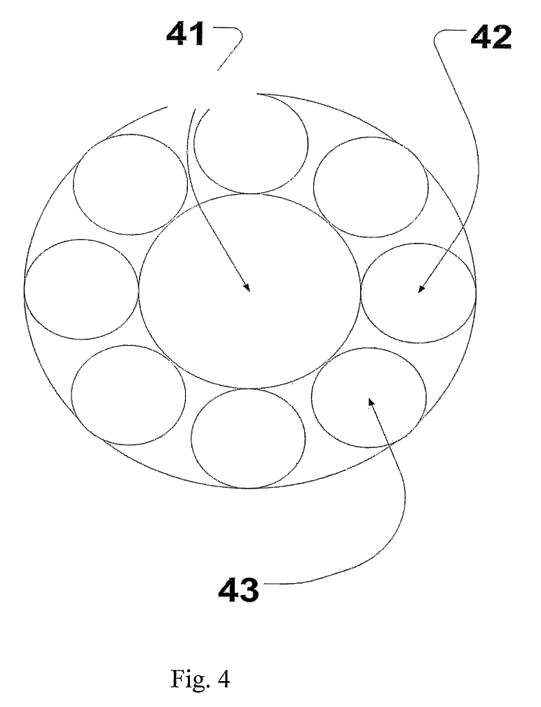

FIG. 4 schematically presents an optic fiber technique for emitting the light to the point and collecting the reflected light for the purpose of sensing it at the other end of the fiber;

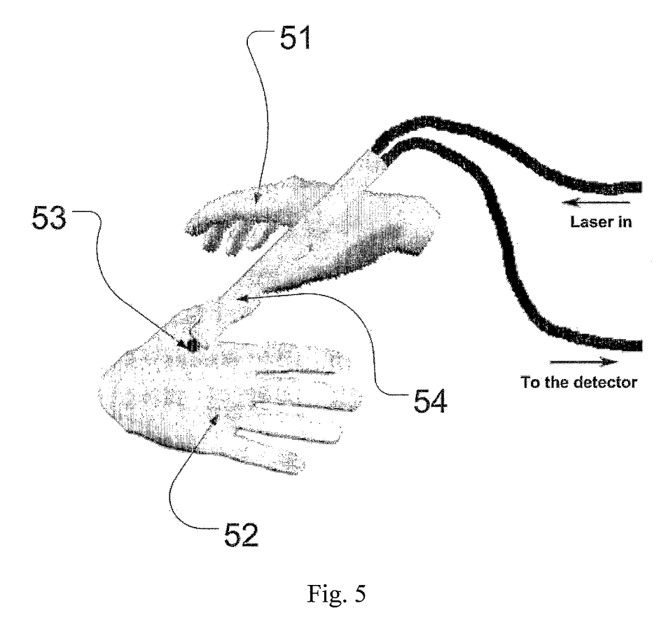

FIG. 5 schematically presents a full view of photonic energy direction device wherein optical fiber technique of FIG. 4 is used fro emission and detection of radiation energy;

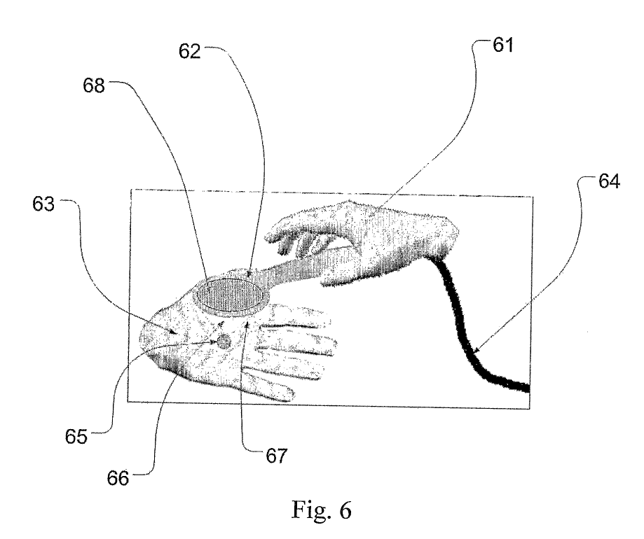

FIG. 6 schematically presents an ultrasound treatment unit;

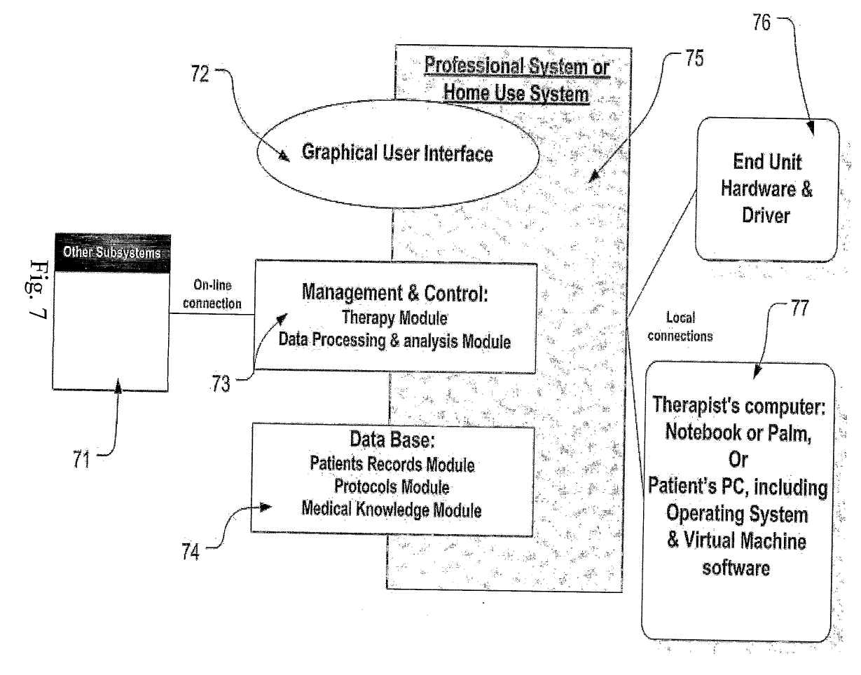

FIG. 7 schematically presents a professional or home use data processing system, characterized by various levels of diagnostic capabilities;

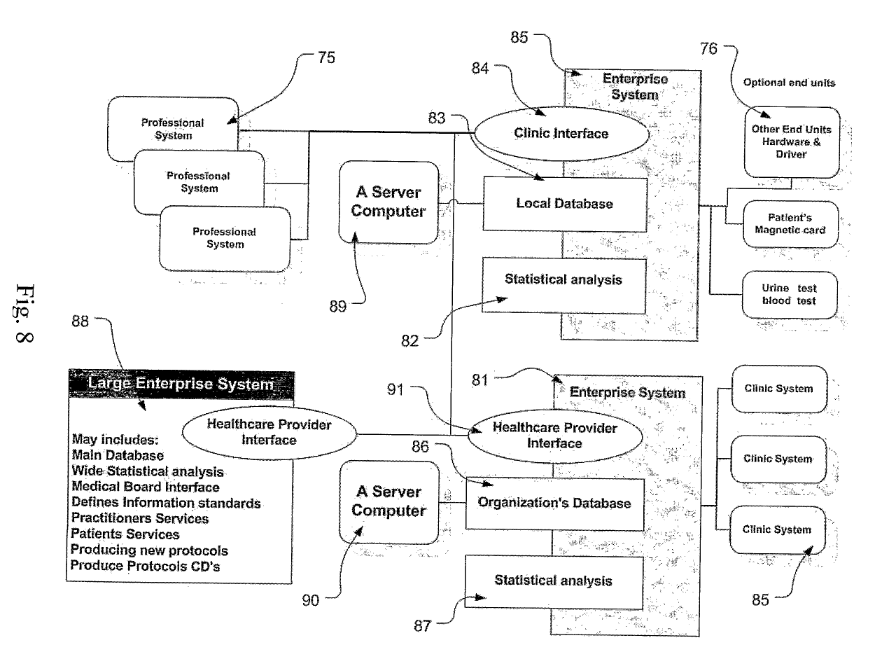

FIG. 8 schematically presents the enterprise system model; for example, healthcare provider system, which is connected to a home or professional system;

FIG. 9 presents an example of the summary graph (TW5 & TW6 in two dimensions) from the extensive mapping of absorption at acupuncture points conducted by MedDynamics Ltd using the method of integrating sphere, and

FIG. 10 presents an example of the summary graph (TW5 & TW6 in three dimensions) from the extensive mapping of absorption at acupuncture points conducted by MedDynamics Ltd using the method of integrating sphere.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a guided medical system for administration of energy-directed acupuncture.

The term ‘acupuncture’ refers in the present invention to any treatment directed at specific locations on the human body, which we refer to as “acu-points” or acupuncture points. Examples of such modes of treatment include: acupuncture, acupressure, traditional Chinese acupuncture, reflexology, application of treatment to so called “trigger points” used in physical and neurological therapy and other practices of point directed therapy, used for systemic medical treatment or for relief of localized pain. These therapies may be carried out through modalities such as needle insertion, electrical stimulation, pressure, heat, light, ultrasound or other such treatments, and may be directed to particular points of the body to treat a specific body part of concern, or portion thereof, either through direct application to the body part or through application to representative therapeutic system points of the body part. The term ‘acupoint’ includes accordingly any acupressure points, acupuncture points, trigger points and meridian points.

The present invention also relates to the communication of medical data as part of treatment administration., whereby the information is communicated by means of a Common Medical Information Protocol (CMIP). The CMIP may include records of information recognized by all elements such as: patient data archiving module; practitioner data archiving module; anamnesis data module; diagnosis protocols data module, diagnosis registration data module; treatment protocols data module; clinical investigation management module; medical knowledge data module; information security data module; registry data module; controlling, monitoring and recording module, data retrieving module and other related modules. The CMIP may further include a header record, which includes a summary about the information packages sent, their size, and any other information.

Reference is made now to FIG. 1, presenting an energy-directed acupuncture system according to a specific embodiment of the present invention in the form of a photonic energy-emitting device (a laser). The therapy is administered by shining the photon beam onto the body of the patient at the acu-points. The photons emanating from the laser (11) are partially absorbed in the tissue and partially scattered. The scattered photons produce a spot (12) on the tissue, such spot being detectable by common photonic sensors (13). The device can be operated in at least two main modes: a treatment mode and a diagnostic mode. In a purely treatment mode the laser is operated at the acu-point known to the therapist. The therapist (who could also be the patient himself in a self-administered mode) holds the applicator part of the device, which is typically in the form of a hand-held stylus (10). The energy (11) emanated from the applicator is controlled by an internal control unit or an external controlling computer (14), which could be in the same locality or a remote location. The controlling unit directs the physician in performing the treatment and applies the appropriate protocol as suggested from a stored database. The protocol includes the indicated treatment items for the disease identified by the therapist. Such items could be the number of sessions needed, suggested points of treatment, the wavelength of light in nanometers to be used the amount of light power in Watts at each point, the length of pulses, their duty cycle (relative on/off time), their frequency in Hertz, the total irradiated energy in Joules and the total delivered energy in Joules to the patient at the chosen point. In the course of the treatment a record of the treatment is generated, including identification of the patient, the diagnosis and the parameters chosen for treatment, some of which are listed above. The record so generated may be stored for the therapist and patient's own records and may additionally further be used in clinical studies of particular diseases and effectiveness of various modes of therapy. Such studies analyzed statistically may yield new or modified protocols. Such protocols may be distributed by a centralized system and used in future therapies.

As part of its treatment mode, the system can also be used to find the optimal location of irradiation near the location of the acu-points determined by the therapist. Such optimal locations are characterized by maximal light absorption capability near the acu-point.

To find the optimal location, a scan is performed over the neighborhood of the acu-point. During scanning the laser device (10) sends a beam (11) at some pre-determined frequency while the spot visible on the skin (12) is sampled by an image sensor such as a CCD or CMOS camera (13).

In the course of the scan an absorption map relating the amount of absorption to the location near the acu-point is displayed on a display device such as computer monitor, enabling the therapist to choose the optimal location for treatment. The map may be computed and drawn according to the method indicated as follows.

The energy of the photons striking the body undergoes scattering, absorption and reflection as schematically presented in FIG. 2. The scattering centers are mitochondria, myofilaments, intracellular matrix, plasma etc. The absorbers of the emitted radiation are haemoglobin, hemoglobin and other globulins, water, fat, proteins, cytochromes, β-carotenes, melamine, glucose etc. The reflected part (i.e., not a random scattering) is relatively small and may range from 2 to 7%. The other components, in the form of coefficients of absorption and scattering, are measured from the sampled photons by means of a known algorithm.

It is known in the art that the rate of absorption of photons at the acupoints is relatively greater than at non-acupoints. The purpose of the scan is to map the optimal location for treatment according to the amount of absorption.

Reference is made now to FIG. 3, schematically presenting a flow chart of one suitable algorithm for computation of the absorption and scattering.

It is possible to use well-known methods such as Video Reflectometry for accurate determination of absorption and scattering of light in-vivo in a non-invasive manner. A photon beam is impinged on the tissue and creates a detectable spot consisting of the scattered photons. The spot is sampled by a CCD or a CMOS sensor. From the sampled image a profile of the spot can be obtained, and displayed as a curve. This curve shows the scattered light as a function of the distance from the center.

From the information embodied in the curve it is possible to compute the coefficients of scattering and absorption of the illuminated volume element, using one of the following two methods, namely Monte Carlo or Farrell model. Monte Carlo simulation is suitable for determining of the propagation of light in the medium in a model positing an a priori unknown amount of random scattering and absorption at each volume element. The simulation starts with an initial estimate of the coefficients. It computes the implied scattered spot, compares it with the observed data, computes the discrepancy and corrects the values of the coefficients in a closed loop- feedback fashion (see steps 31-37 at FIG. 3). Instead of such a simulation it is possible to compute an approximation using a model based on the diffusion type model of Farrell et al. as described in Phys. Med. Biol. 37: 2281-2286 (1992). This diffusion model is approximate and is based on a model with simple boundary conditions in a simplified geometry.

The Monte-Carlo method is iterative and converges to a solution in a few cycles as described at FIG. 3 in any geometry. The solution obtained by the diffusion model can be used as a first approximation for the Monte Carlo solution.

The device so described may also be used in a diagnostic mode. In such a mode the therapist will be guided by the system according to predefined diagnostic protocols. As part of operation in this mode, the optimal locations will first be found and the absorption measured before treatment. For diagnosis as well as treatment progress monitoring, the amount of absorption at indicated points will also be recorded over several treatment sessions and will be included as part of the treatment report. It is known that the amount of absorption may yield diagnostic information on the patient's condition as well as on the progress of healing.

Reference is made now to both FIGS. 4 and 5 also depicting a device based on light emitting principles, however the transmission of light radiation to the body and monitoring of its effect is undertaken by means of fiber optics and not by propagating the light in free space.

In such a unit the light is applied by means of an optical fiber whose cross section is seen in FIG. 4. The incident light may be propagated from a laser through a central fiber in a bundle of such fibers (41) while the emerging light scattered from the tissue may be collected and transmitted to a sensor at the other end by means of other fibers (42, 43 and other peripheral fibers for example).

The method of application is further illustrated in FIG. 5. There the therapist's hand (51) is shown holding the end-piece of such a bundle (54) just above a treatment point (53). An incoming part of the fiber bundle carries the incident light from a source such as a laser onto the application end while the other part of the bundle carries the emerging light toward a detector.

Another embodiment of a directed energy acupuncture system uses ultra sound energy as described in FIG. 6: In such a device the therapist's hand (61) holds an applicator that is capable of transmitting sound energy at ultrasound frequencies. The shape of the transmitter determines the focal point of such transmittance at some distance below its structure. A soft material (67) designed to have propagation parameters of such sound waves similar to that of human tissue is attached below the transmitter and permits the reposing of the transmitter so that its focus is at a desired location on a treatment point (65) (which could be an acupuncture point). The waves propagate inside a cone (67) and are enabled by the construction of the transmitter to arrive at the treatment point (65). An ultrasound sensor (68) (such as available commercially for ultrasound imaging) is situated inside the applicator and provides imaging of the ultrasound beam for control of the procedure. Its image is transmitted via a cable (64) to a computer (not shown). The computer also controls the application of the sound energy according to the CMIP.

Other embodiments of directed energy systems are possible, and may consist of therapy-delivery modules that may be converted to end-units as defined below in a non-limiting manner: thermo module for local heating or cooling, which may be used for physiotherapy treatment and for measurement or diagnosis using a thermometer; applicator for applying chemical agents to the skin to be used for treatment while measurement or diagnosis may consist of perspiration detection; biofeedback systems available on the market that include a measurement capability ; brain electric potential measurement systems that may be used for measurement or diagnosis, such as Low Resolution Brain Electromagnetic Tomography, (LORETA) system, which is used to analyze E.E.G. output data for locating of current brain activity, as presented by R. D. Pascual-Marqui, M. Esslen, K. Kochi, D. Lehmann “Functional imaging with low resolution brain electromagnetic tomography (LORETA): a review” in Methods & Findings in Experimental & Clinical Pharmacology 2002, 24C:91-95; and analysis modules, such as glucose-meter systems that are preferably used for measurement and study. There are also glucose-meter devices available also for treatment by Insulin injection, but only as stand alone devices and not connected online as an end-unit of a main system; Light and sound therapy systems, pulsed or continuous are used for therapy but not yet for measurement or diagnosis, magnetic field applicators used for acupuncture treatment and other therapies but not yet as a stand alone measurement or diagnosis device or any combination thereof.

All the above examples of end-units have in common the characteristics of allowing the system to interact with the human body in terms of input/output and by doing so the information gathered by the system can be made to improve actual treatment and contribute to the medical knowledge shared among professionals.

For the data coming out of and flowing into the end-unit to be monitored, shared and distributed, a hierarchical set of higher-end units may be employed. Each such higher-end unit may be a system such as a computer, communicable by CMIP to the end unit and possible higher-units, and able to store, process, analyze and distribute medical data.

Following is a description of home, professional and enterprise units.

The present invention allows medical data file exchange: the patient or the physician can record medical information or data with a properly interfacing end-unit, format it as a standard digital file (e.g., CMIP) and exchange it with other users. This exchange allows creation of summary data thereby contributing to general medical knowledge that in turn permits individual professionals to provide better treatment to their patients. Moreover, a home use system provides a safe device for treatment by the non-professional user, and allows the patient to treat medical problems at home when these problems are minimal. By doing so the system provides more efficient medicine, with a preventive aspect, and better cost-efficiency for a global medical system. The patient has the ability to connect to a main database to obtain medical data and thus improve self-care opportunities.

Such a home use system, as described in FIG. 7, may be used by a private professional or by a patient for self administration of treatment, and includes a personal computer (77), an End Unit as described above (76), which might be connected to the system (75) wirelessly with, for example, Bluetooth technology; on-line connection to other systems (71), such as a symmetric digital subscriber line (ADSL) fast cable connection, or a cellular connection such as CDMA Technology; and management software (72, 73, 74), for example community edition software, that includes modules such as graphical user interface (72), management & control (73) (Therapy Module, Data Processing & Analysis Module), Local Data Base (74) (Patients Records Module, Protocols Module, Medical Knowledge Module). The protocols module includes a diagnostic module, and a treatment module. Both types of protocols may be updated on-line when needed.

The professional system may include (as detailed at FIG. 7): a personal computer (77) such as IBM® ThinkCenter, with an operating system such as one of Microsoft® Windows® products, or a free-to-use Linux® distribution. It might use third party software such as SUN® Javaplatform®, or Microsoft® .NET® platform and CLR® software; an End Unit (76) as described above, that might be connected to the system wirelessly with, for example, Bluetooth technology.

On the other hand, a professional system (75) used by medical practitioners may also be used in a Healthcare Provider Enterprise System constructed from a number of subsystems.

The Healthcare Provider Enterprise System, as described at FIG. 8, is constructed from at least one professional system (75), which is connected to a clinic system (85), while at least one clinic system is connected to the Healthcare Provider Enterprise System. The connections between those subsystems might be on line, such as ADSL fast cable connection, or a cellular connection such as CDMA technology. The enterprise system used at a clinic (85) is constructed from a clinic server computer (89) with Clinic Interfacing Software (84), a local database (83) and a statistical analysis module (82). One or more end units (76) are capable of being connected directly to the clinic system, these are end units, which serve a larger number of patients, for blood and urine tests for example.

At lesst one clinic system (85) is connected to an Enterprise system used as a healthcare provider System (81), which uses Healthcare Provider Interface Software (91) on a server computer (90), and has its own database (86) and its own module for data analysis (87).

The Healthcare provider System (81) might be connected to a large Enterprise System (88), which might be a governmental or international organization, (World Health Organization for example). The large system might have its own Healthcare Provider Interface Software, server, database and statistic analysis modules, and might use its medical board to define Medical Information Standards that the invented system will use as CMIP.

1. A system for administering medical therapy by means of directing energy radiation onto a patient's body; said system inter alia comprising:
a. an assembly for directing said energy in a predetermined measure comprising means for emitting sufficient energy to be directed toward the patient's body, and communication means;
b. a treatment protocol defining a set of parameters according to which the emitted energy radiation is to be delivered, such parameters include predetermined points on the patient's body whereat the energy is to be directed; and,
c. a system for controlling said emitted radiation according to said treatment protocol.

2. A system according to claim 1 further comprising a means for monitoring the energy absorbed by the tissue at each application at a treatment point

3. A system according to claim 2 wherein the treatment points are acupuncture points.

4. A system according to claim 3 wherein the emitted energy radiation is light and the monitoring device is a light detecting sensor or an imaging sensor.

5. A system according to claim 3 wherein the emitted energy radiation is ultrasound energy and the monitoring device is an ultrasound wave sensor.

6. The system according to claim 1 or any of its dependent claims, wherein the protocol module consists of a Common Medical Information Protocol (CMIP) used to communicate between the energy directing module and the system and possibly to communicate with other medical systems; wherein said system and module are adapted to emit directed energy at points in the body as guided by said CMIP so that anamnesis, diagnosis and targeted treatment is provided, monitored, and/or recorded.

7. The system according to claim 6, wherein the CMIP's module is selected from the group of: patient data archiving module; practitioner data archiving module; anamnesis data module; diagnosis protocols data module, diagnosis registration data module; treatment protocols data module; clinical investigation management module; medical knowledge data module; information security data module; registry data module; controlling, monitoring and recording module, data retrieving module or any combination thereof.

8. The system according to claim 6, additionally comprising a unit for analyzing the detectable image of the irradiated spot said analysis used to determine parameters of the incident radiation and its effect on the body at a treatment point and/or measure the clinical state of a treatment point.

9. The system according to claim 6 where the measured radiation parameters are used to optimally locate the point for treatment near or at the estimated location of the acupuncture point

10. The system according to claim 6, additionally comprising software adapted to collect the data of treatment points and analyze the data so as to produce a medical diagnosis.

11. The system according to claim 6, additionally comprising software adapted to use a treatment protocol for performing treatment according to a defined diagnosis.

12. The system according to claim 6, additionally comprising a unit to collect the therapy data for future review, and for statistic analysis.

13. The system according to claim 6, additionally comprising software for performing statistical analysis as to improve information contained in CMIP's data modules

14. The system according to claim 6, additionally comprising software for analyzing the collected data according to widely accepted Clinical Investigation Standard, to augment the reliability of CMIP and especially the treatment and diagnosis protocols

15. The system according to claim 6, wherein the energy directing means is a laser assembly comprising a bundle of fibers is adapted to emit light from a light source while the energy detecting means is performed by conducting the light scattered by the tissue onto another part of the bundle so as to measure it by a light sensor located on its end furthest away from the tissue.

16. A method for administration of laser acupuncture by means of a medical device comprising an emitter of photonic radiation directed to an acupuncture point and an imaging sensor for monitoring and controlling the treatment.

17. The method according to claim 16, comprising the steps of locating the acupuncture point; measuring the photo-absorption of an acupuncture point; determining its functionality, and applying laser treatment to said point.

18. The method according to claim 16, further comprising the steps of communicating with a database, so that data archiving and/or data retrieving is achieved.

19. The method according to claim 16, further comprising the step of collecting the data of treatment points and analyzing the data so as to produce a medical diagnosis.

20. The method according to claim 16, further comprising the step of using a defined diagnosis for performing a treatment according to a treatment protocol;

21. The method according to claim 16, further comprising the step of collecting the therapy data for future review, and for statistic analysis.

22. The method according to claim 16, further comprising the step of performing statistical analysis for clinical investigation of diagnosis and treatment protocols.

23. The method according to claim 16, further comprising the step of performing statistical analysis so as to improve the diagnosis and treatment protocols.

24. The method according to claim 16, further comprising the step of analyzing the collected data according to widely accepted Clinical Investigation Standard to augment the reliability of treatment and diagnosis protocols.

25. The method according to claim 16, further comprising the step of analyzing light scattered from the tissue for location of treatment point and/or measuring the clinical state of a treatment point.
